(19) United States
(12) Patent Application Publication
(10) Pub. No.: US 2003/0153486 A1
(43) Pub. Date: Aug. 14, 2003

(54) Method for obtaining perfuming compositions and perfumed products and resulting products

(76) Inventor: Isabelle Storet, Les Eparres (FR)

Correspondence Address: Kevin McVeigh, Intellectual Property Department, Rhodia Inc CN 7500, 259 Prospect Plains Road, Cranbury, NJ, 08512, US

(21) Appl. No.: 10/221,715

(22) PCT Filed: Mar. 13, 2001
(86) PCT No.: PCT/FR01/00744

(30) Foreign Application Priority Data
Mar. 13, 2000 (FR) 00/03175

(51) Int. Cl. A61K 7/46

(52) U.S. Cl. 512/25

(57) ABSTRACT

The invention concerns a method for obtaining perfuming compositions, perfumed substances and perfumed products for perfumery, characterized in that they contain, as active principle imparting smell, an efficient amount of a 2-hydroxy-3-butenoic acid ester.

The present invention relates to a process for the production of perfuming compositions, to perfumed products, and to products so obtained.

In particular, the present invention envisages their use in the perfumery field. Such compounds exhibit very interesting olfactory properties and can be used, inter alia, to prepare perfuming compositions and perfumed products.

The perfumery industry is constantly seeking products which by their originality, volume and the strength of their fragrance can endow compositions containing them with a unique character.

We have now discovered that 2-hydroxy-3-butenoic acid esters as defined below exhibit novel olfactory properties.

It should be noted that it is impossible for the skilled person to predict whether a given chemical compound will or will not have an interesting scent from the olfactory viewpoint which could be used in the perfumery field.

More specifically, the present invention concerns a process for the production of perfuming compositions, perfumed products and substances for perfumery, characterized in that an effective quantity of a 2-hydroxy-3-butenoic acid ester is added to the usual constituents of these compositions, substances and finished products.

The present invention also concerns perfuming compositions, perfumed substances and products characterized in that they comprise, as an active ingredient having an influence on the scent, an effective quantity of a 2-hydroxy-3-butenoic acid ester.

The invention thus resides in a novel use of esters of a 2-hydroxy-3-butenoic acid as a perfuming ingredient.

The perfuming ingredient of the invention is a 2-hydroxy-3-butenoic acid ester. More particularly, it has the following general formula (I):

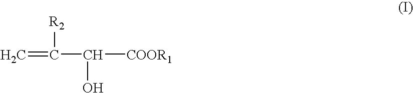

in which formula (I):

R₁ represents a radical, which may or may not be substituted, containing 1 to 40 carbon atoms, more particularly a linear or branched, saturated or unsaturated, aliphatic acyclic radical; or a saturated or unsaturated, monocyclic or polycyclic, carbocyclic or heterocyclic radical;

R₂ represents an aliphatic radical, which may be saturated, with a low carbon condensation, preferably less than 4.

In the following description, the expression “hydroxybutenoic ester” is used generically to designate all compounds with formula (I).

More particularly, hydroxybutenoic esters have formula (I) in which the different radicals R₁ and R₂ have the following meaning.

The number of carbon atoms in R₁ is generally between 1 and 40 carbon atoms, preferably between 1 and 12 carbon atoms.

Radical R₁ can be a monovalent radical which may or may not be substituted, which may be a linear or branched, saturated or unsaturated, aliphatic acyclic radical; or a saturated or unsaturated, monocyclic or polycyclic, carbocyclic or heterocyclic radical.

Radical R₁ is a linear or branched, saturated or unsaturated, aliphatic acyclic radical.

More precisely, R₁ is a linear or branched alkyl, alkenyl or alkadienyl radical, preferably containing 1 to 40 carbon atoms.

The hydrocarbon chain can optionally be interrupted by a heteroatom (for example oxygen or sulphur) or by one of the following groups: —CO—, —COO—, and/or can carry one of the following substituents:

—OH, —COOR₃: in these formulae, R₃ preferably represents hydrogen or a linear or branched alkyl radical containing 1 to 4 carbon atoms, more particularly a methyl or ethyl radical.

R₁ can represent a carbocyclic monocyclic radical. The number of carbon atoms in the cycle can vary from 3 to 8 carbon atoms, but is preferably 5 or 6 carbon atoms.

The carbocycle can be saturated or can contain 1 or 2 unsaturated bonds in the cycle, preferably 1 or 2 double bonds.

Examples of carbocycles are a cycloalkoyl or cycloalkenyl radical containing 3 to 8 carbon atoms, preferably a cyclohexyl, cyclohexen-yl or cyclohepten-yl radical.

When R₁ represents a saturated or unsaturated carbocyclic monocyclic radical, it is possible for one or more carbon atoms of the cycle to be replaced by one or more heteroatoms, preferably oxygen, nitrogen or sulphur, or by a functional group, preferably a carbonyl or ester group, resulting in a monocyclic heterocyclic compound. The number of atoms in the cycle can vary from 3 to 8 atoms but is preferably 5 or 6 atoms.

Radical R₁ can also be carbocyclic and polycyclic, preferably bicyclic, which means that at least two cycles have two carbon atoms in common. For polycyclic radicals, the number of carbon atoms in each cycle is between 3 and 6: the total number of carbon atoms is preferably 7.

Examples of currently encountered bicyclic structures are given below:

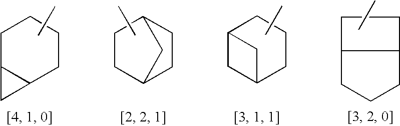

Radical R₁ can also be heterocyclic and polycyclic, preferably bicyclic, meaning that at least two cycles have two atoms in common. In this case, the number of atoms in each cycle is between 3 and 6, more preferably 5 or 6.

Radical R₁ can represent a linear or branched, saturated or unsaturated aliphatic radical carrying a cyclic substituent. Examples of cyclic substituents are cycloaliphatic, aromatic or heterocyclic substituents, in particular cycloaliphatic compounds containing 6 carbon atoms in the cycle, or benzene rings. More particular examples that can be cited are arylalkyl radicals containing 6 to 12 carbon atoms, preferably the benzyl or β-phenylethyl radical.

It should be noted that if radical R₁ contains a cycle, it is possible for the cycle to carry a substituent of any type. The substituents usually carried by the cycle are one or more alkyl or alkoxy radicals preferably containing 1 to 4 carbon atoms, preferably three methyl radicals, a methylene radical (corresponding to an exocyclic bond), an alkenyl radical, preferably an isopropen-yl radical, or a halogen atom, preferably chlorine or bromine.

More preferably, the alkylsalicylic acid esters have the following general formula (Ia):

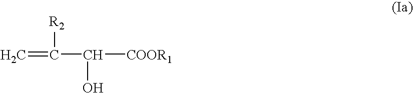

in which formula (Ia):

R₁ represents a linear or branched alkyl radical containing 1 to 12 carbon atoms, preferably 1 to 6; or a cycloalkyl radical preferably containing 6 carbon atoms, or an aralkyl radical containing 6 to 12 carbon atoms, preferably 7 or 8 carbon atoms;

R₂ represents a linear or branched alkyl radical containing 1 to 4 carbon atoms, preferably 1 or 2.

Preferred compounds are those with formula (Ia) in which R₁ represents an alkyl radical such as methyl, ethyl, n-propyl, isopropyl, butyl, isobutyl, sec-butyl, tert-butyl, amyl, isoamyl, n-hexyl; an allyl radical, or a 2-hexenyl radical; a cyclohexyl radical; or a benzyl radical or a 13-phenylethyl radical, and R₂ represents a methyl or ethyl radical.

Specific examples of hydroxybutenoic acid esters with formula (I) that can be mentioned include the methyl ester of 2-hydroxy-3-methyl-3-butenoic acid or the ethyl ester of 2-hydroxy-3-methyl-3-butenoic acid.

Compounds with formula (I), in particular the ethyl ester of 2-hydroxy-3-methyl-3-butenoic acid have an autumnal ambience and are reminiscent of the aroma of moist undergrowth enriched with fruity nutty notes and of apple compote.

These products can be used as perfuming ingredients in perfuming compositions, substances and perfumed products.

The term “perfuming compositions” means mixtures of various ingredients such as solvents, solid or liquid supports, fixatives, various scenting compounds, etc. . . . , into which 2-hydroxy-3-butenoic acid esters with formula (I) are incorporated, which are used to produce a variety of types of finished products with the desired fragrance.

Perfume bases constitute preferred examples of perfuming compositions in which the 2-hydroxy-3-butenoic acid esters with formula (I) can advantageously be used.

Eau de toilette, after-shave lotion, perfume, soap, bath or shower gel or deodorant or antiperspirant products in the form of sticks or lotions constitute examples of finished products which the 2-hydroxy-3-butenoic acid esters with formula (1) endow with their original note.

They can also be used in all types of shampoos and hair-care products.

They can also perfume all types of talcs or powders.

They can also be used in room sprays or any cleaning product.

The amount in the compositions of the invention of 2-hydroxy-3-butenoic acid ester with formula (I), expressed as the percentage by weight in the composition under consideration, depends on the nature of the composition (a base for a perfume or eau de toilette, for example) and the strength and nature of the desired influence in the finished product. It is clear that in a perfume base the quantity of 2-hydroxy-3-butenoic acid ester with, formula (I) can be very high, for example over 50% by weight, and can attain 90% by weight while in a perfume, an eau de toilette or an after-shave lotion, this quantity could be much less than 50% by weight.

Thus, the lower limit of the amount of 2-hydroxy-3-butenoic acid ester with formula (I) can be that which causes a perceptible modification in the scent or fragrance or the note of the finished product. In some cases, this minimum amount can be of the order of 0.01% by weight. Clearly, quantities which are not included in the limits indicated above can be employed without departing from the scope of the invention.

It can also be used in perfumed shampoos in an amount of 0.5% to 2%, or to perfume any hair product.

One particular application of 2-hydroxy-3-butenoic acid esters as a perfume is in the soap industry.

Soap is essentially a mixture of alkali metal salts, preferably sodium or potassium salts of saturated or unsaturated long chain fatty acids generally containing 8 to 24 carbon atoms, preferably 12 to 18 carbon atoms.

Examples of fatty acids that can be cited are saturated fatty acids such as caprylic acid C₈, capric acid C₁₀, lauric acid C₁₂, myristic acid C₁₄, palmitic acid C₁₆, stearic acid C₁₈, unsaturated fatty acids with a single double bond such as linderic acid C₁₂, myristoleic acid C₁₄, palmitoleic acid C₁₆, oleic acid C₁₈; unsaturated fatty acids with 2 double bonds such as linoleic acid C₁₈; unsaturated fatty acids with 3 double bonds such as linolenic acid C₁₈; unsaturated fatty acids carrying a hydroxyl group such as ricinoleic acid C18, and mixtures thereof.

Soap is prepared by saponifying triglycerides derived from a variety of fats, for example tallow, peanut oil, palm oil, coconut oil, olive oil, etc. . . .

The fat mixture is chosen on the basis of economic considerations and desired characteristics.

A conventional method for producing soap is the “Marseilles” process comprising the following steps: saponification with sodium hydroxide, heating, washing and liquidation, which puts the soap into a physical form that can readily be used subsequently.

After those steps, the liquid soap is transformed into pieces for use by the consumer. This latter phase, known as finishing, comprises the operations of drying, mixing, extrusion, cutting, stamping and packing.

During mixing, a certain number of ingredients are added which provide the product with its features: talc, fillers, colorants, sequestrating agents, deodorants, fragrances. 2-hydroxy-3-butenoic acid esters can be added at this stage of the production process.

The amount of 2-hydroxy-3-butenoic acid ester that can be used in soaps is of the order of 1% to 2%.

A further example of compositions into which said compounds can advantageously be introduced is represented by the usual detergent compositions for washing laundry (industrial or domestic washing).

Such compositions generally comprise one or more of the following ingredients: anionic, cationic or amphoteric surfactants, bleaching agents, optical brighteners, various fillers, anti-redepositing agents.

They generally comprise at least one surfactant in a quantity that is generally of the order of 5% to 60% by weight, preferably 8% to 50% by weight. Preferred compounds that can be cited are anionic or non-ionic surfactants that are normally used in detergents for laundry washing.

They can also comprise the usual additives, such as mineral or organic builders, in a quantity such that the total quantity of detergent additive is of the order of 5% to 80% of the composition weight, preferably 8% to 40% by weight, anti-soiling agents, anti-redepositing agents, bleaching agents, fluorescent agents, anti-foaming agents, softeners, enzymes and other additives.

The perfuming ingredient of the present invention, namely a 2-hydroxy-3-butenoic acid ester, can be used in an amount of about 0.5% to 2.5%, preferably 1% to 2% by weight with respect to the detergent composition.

The nature of the various components of the detergent composition is not critical and 2-hydroxy-3-butenoic esters with formula (I) can be added to any detergent composition type. They can be introduced into fabric softeners, in the form of a liquid or into compositions deposited onto a support, usually a nonwoven support, for use in clothes dryers.

The 2-hydroxy-3-butenoic acid esters used in accordance with the invention as perfuming ingredients are known products that have been described in the literature.

For their synthesis, reference should be made to the articles by Burr C. Hartmann et al. [J. Org. Chem. 57, n 7, p. 943-946 (1972)] and E. Voget et al. [Helvetica Chimica Acta 33, 1, n° 19, p. 119 (1950)].

One prepared preparation mode, which is illustrated in the example, consists of reacting:

a ketone with formula (II):

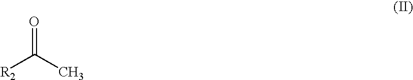

in which R₂ has the meaning given above;

with a halogenoacetic acid ester with formula (III):

XCH₂—COOR₁ (III)

in which X represents a bromine or chlorine atom and R₁ has the meaning given above.

The reaction takes place in the presence of a strong base, in particular sodium or potassium methylate or ethylate.

It is carried out at a temperature of 0° C. to ambient temperature (usually between 0° C. and 25° C.).

The reaction is advantageously carried out in the presence of an organic solvent that dissolves the product obtained and thus enables it to be separated from the reaction medium.

Preferred solvents that can be used are aliphatic or aromatic hydrocarbons, more particularly benzene, toluene, xylenes or alkylbenzene type petroleum cuts. Advantageously, toluene is selected.

The product recovered from the organic phase has formula (IV):

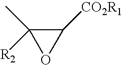

in which R₁ and R₂ have the meanings given above.

The 2-hydroxy-3-butenoic acid ester is obtained by isomerising the compound with formula (IV).

This operation is carried out by heating the organic phase comprising the compound with formula (IV).

The reaction temperature is between 100° C. and 150° C.

The reaction is preferably carried out at atmospheric pressure.

The compound with formula (I) is obtained, which is separated from the organic phase in a conventional manner, in particular by distillation.

The 2-hydroxybutenoic acid ester obtained can advantageously be used in the perfuming compositions of the invention.

An illustrative, non-limiting example of the invention will now be given.

In Example 1, the abbreviation EHMB means the “ethyl ester of 2-hydroxy-3-methylbutenoic acid”.

### EXAMPLE 1

1—In this example, the ethyl ester of 2-hydroxy-3-methylbutenoic acid (EHMB) was prepared using a process that consisted of isomerising ethyl dimethylglycidate.

a-Preparation of Ethyl Dimethylglycidate

756 g (10 moles) of sodium ethylate and 51 of toluene were charged into a flask.

It was cooled to 5° C. and a mixture constituted by 639 g (11 moles) of acetone and 1226 g (10 moles) of ethyl chloroacetate was added dropwise over a period of 4 hours.

The reaction mixture was cooled to ambient temperature and stirred for 12 h.

51 of water was added.

It was decanted.

The aqueous phase was washed twice with 11 of toluene.

The organic phases were combined and the resulting phase was washed with 31 of water.

The organic phase was dried over magnesium sulphate.

b-Preparation of EHMB

The toluene solution prepared above was heated at 110° C. for 3 hours in the presence of 1.5 g of p-toluenesulphonic acid.

It was cooled then neutralised with sodium bicarbonate.

The toluene was eliminated under 130 mbars. The ethyl 2-hydroxy-3-methyl-3-butenoate was distilled under 10 mbars (boiling point 68° C.).

A colourless liquid was recovered in a yield of 55% for the two steps. 2-EHMB has a powerful head note

It re-creates an autumnal ambience and is reminiscent of the moist scent of plum-tree evernia or an oak cask that has matured good wine.

The woody richness is enriched by the nutty notes emanating from it.

A fruity pear compote facet enlivens the olfactory bouquet of this molecule.

### EXAMPLE 2

1.5 g of a 33% by weight solution of ethyl 2-hydroxy-3-methyl-3-butenoate in polysorbate 20 (ester of sorbitol and fatty acids (lauric, stearic, oleic) ethoxylated with 20 OE units) was incorporated into 98.5 g of a shampoo base.

The shampoo had the following composition by weight:

| | |
|---|---|
| Sodium lauryl sulphate + ethoxylated sodium lauryl | 30% |
| sulphate + disodium cocoamphodiacetate + hexylene | |
| glycol (MIRACARE 2MCA S/E) | |
| Cocamidopropylamine oxide and lichen extract | 1% |
| (ANTIPELLICULE USINATE AO) | |
| Indian cress extract (CAPUCINE HS) | 1% |
| Preservative (GERMABEN II) | 0.2% |
| Citric acid | qsp pH 6.0 to 6.2 |
| Demineralised water | 67.3% |
| Perfume | 0.5% |

The perfumed shampoo was stable as regards colour and scent for a period of 3 months when it was stored at 50° C. away from light and when it was stored at 20° C. in daylight.

### EXAMPLE 3

1.5 g of a 33% by weight solution of ethyl 2-hydroxy-3-methyl-3-butenoate in polysorbate 20 was incorporated into 98.5 g of a shower gel base.

The shower gel had the following composition by weight:

| | |
|---|---|
| Ethoxylated sodium lauryl sulphate + sodium | 37.30% |
| cocoamphodiacetate + MIPA cocamide (MIRACARE CS) | |
| Wild camomile HS | 2.00% |
| Hydroxypropylated guar (JAGUAR C 162) | 0.30% |
| Preservative (GERMABEN IIE) | 0.20% |
| Demineralised water | 59.7% |
| Citric acid | qs pH 5.9 |
| Perfume | 0.50% |

The perfumed shower gel was stable as regards colour and scent for a period of 3 months when it was stored at 50° C. away from light and when it was stored at 20° C. in daylight.

### EXAMPLE 4

0.3 g of ethyl 2-hydroxy-3-methyl-3-butenoate was incorporated into 99.7 g of a moisturising cream base.

The moisturising cream base was prepared by mixing two phases with the following composition by weight:

| Phase A | |
|---|---|
| Capryl/capric triglyceride (DERMOL M5) | 4% |
| Mineral oil (MARCOL 82) | 2% |
| Stearyl alcohol | 3% |
| Isopropyl myristate (WICKENOL 111) | 2% |
| Glyceryl stearate + polyethylene glycol 100 (ARLACEL 165) | 6% |
| Dimethicone (MIRASIL DM 300) | 4% |
| Phase B | |
| Deionised water | 70.7% |
| Glycerol | 8% |
| Preservative (GERMABEN II) | 0.3% |

The perfumed moisturising cream was stable as regards colour and scent for a period of 3 months when it was stored at 50° C. away from light and when it was stored at 20° C. in daylight.

### EXAMPLE 5

0.2 g ethyl 2-hydroxy-3-methyl-3-butenoate was incorporated into 99.8 g of a detergent powder.

The detergent powder had the following composition by weight:

| | |
|---|---|
| Linear sodium alkylbenzenesulphonate (LABS NANSA) | 10% |
| SOAP | 5% |
| Ethoxylated C12 alcohol, 7OE (SINPERONIC A7) | 2% |
| Sodium tripolyphosphate (RHODIAPHOS HPA 3.5) | 25% |
| Sodium carbonate | 10% |
| Sodium silicate R2 | 5% |
| Sodium sulphate | qsp% |
| Sodium carboxymethyl cellulose | 1% |
| Sodium perborate | 15% |
| TAED | 5% |
| Diethylenetriaminepenta(methylenephosphonic acid) | 1% |
| (DEQUEST 2066) | |
| Anti-foaming agent (RHODORSIL 20448) | 1% |
| Optical brightener (TINOPAL DMS) | 0.2% |

The perfumed shampoo was stable as regards colour and scent for a period of 3 months when it was stored at 50° C. away from light and when it was stored at 20° C. in daylight.

### EXAMPLE 6

A perfumed soap was prepared by incorporating 10 g of ethyl 2-hydroxy-3-methyl-3-butenoate into 1000 g of soap extrudates formed by successive passages through an extruder.

The perfumed soap was stable as regards colour and scent for a period of 3 months when it was stored at 50° C. away from light and when it was stored at 20° C. in daylight.

1. A process for the production of perfuming compositions, perfumed products and substances for perfumery, characterized in that an effective quantity of a 2-hydroxy-3-butenoic acid ester is added to the usual constituents of said compositions, substances and finished products.

2. A process according to claim 1, characterized in that the 2-hydroxy-3-butenoic acid ester has general formula (I): 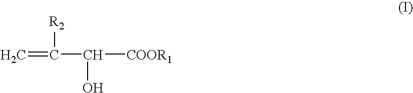
in which formula (i):
R₁ represents a hydrocarbon radical, which may or may not be substituted, containing 1 to 40 carbon atoms, more particularly a linear or branched, saturated or unsaturated aliphatic acyclic radical; or a saturated or unsaturated, monocyclic or polycyclic, carbocyclic or heterocyclic radical;
R₂ represents a saturated aliphatic radical with a low carbon condensation, preferably less than 4.

3. A process according to claim 1 or claim 2, characterized in that the 2-hydroxy-3-butenoic acid ester has general formula (Ia): 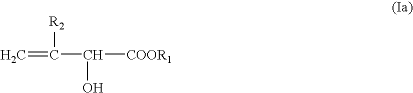
in which formula (Ia):
R₁ represents a linear or branched alkyl radical containing 1 to 12 carbon atoms, preferably 1 to 6; or a cycloalkyl radical preferably containing 6 carbon atoms, or an aralkyl radical containing 6 to 12 carbon atoms, preferably 7 or 8 carbon atoms;
R₂ represents a linear or branched alkyl radical containing 1 to 4 carbon atoms, preferably 1 or 2.

4. A process according to one of claims 1 to 3, characterized in that the 2-hydroxy-3-butenoic acid ester has general formula (I) in which R₁ represents an alkyl radical such as methyl, ethyl, n-propyl, isopropyl, butyl, isobutyl, sec-butyl, tert-butyl, amyl, isoamyl, n-hexyl; an allyl radical; a 2-hexenyl radical; a cyclohexyl radical; or a benzyl radical or a β-phenylethyl radical, and R₂ represents a methyl or ethyl radical.

5. A process according to one of claims 1 to 4, characterized in that the 2-hydroxy-3-butenoic acid ester is the methyl or ethyl ester of 2-hydroxy-3-methyl-3-butenoic acid.

6. Perfuming compositions, substances and perfumed products, characterized in that they comprise an effective quantity of a 2-hydroxy-3-butenoic acid ester as an active ingredient having an influence on scent.

7. Compositions according to claim 6, characterized in that the 2-hydroxy-3-butenoic acid ester has general formula (I): 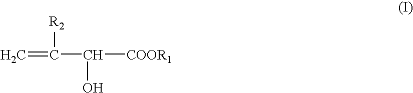
in which formula (I):
R₁ represents a hydrocarbon radical which may or may not be substituted, containing 1 to 40 carbon atoms, more particularly a linear or branched, saturated or unsaturated aliphatic acyclic radical; or a saturated or unsaturated, monocyclic or polycyclic carbocyclic or heterocyclic radical;
R₂ represents a saturated aliphatic radical with a low carbon condensation, preferably less than 4.

8. Compositions according to claim 6 or claim 7, characterized in that the 2-hydroxy-3-butenoic acid ester has general formula (Ia): 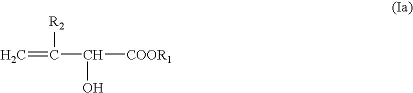
in which formula (Ia):
R₁ represents a linear or branched alkyl radical containing 1 to 12 carbon atoms, preferably 1 to 6; or a cycloalkyl radical preferably containing 6 carbon atoms, or an aralkyl radical containing 6 to 12 carbon atoms, preferably 7 or 8 carbon atoms;
R₂ represents a linear or branched alkyl radical containing 1 to 4 carbon atoms, preferably 1 or 2.

9. Compositions according to one of claims 6 to 8, characterized in that the 2-hydroxy-3-butenoic acid ester has general formula (I) in which R₁ represents an alkyl radical such as methyl, ethyl, n-propyl, isopropyl, butyl, isobutyl, sec-butyl, tert-butyl, amyl, isoamyl, n-hexyl; an allyl radical; a 2-hexenyl radical; a cyclohexyl radical; or a benzyl radical or a β-phenylethyl radical, and R₂ represents a methyl or ethyl radical.

10. Compositions according to one of claims 6 to 9, characterized in that the 2-hydroxy-3-butenoic acid ester is the methyl or ethyl ester of 2-hydroxy-3-methyl-3-butenoic acid.

11. A perfumed article in the form of a perfume, eau de toilette, after-shave lotion, fragrance, soap, bath or shower gel, deodorant or antiperspirant product, shampoo or any hair-care product, talc or powder of any nature, room spray, any cleaning product or detergent product, or fabric softener, characterized in that it comprises at least one 2-hydroxy-3-butenoic acid ester as defined in one of claims 6 to 10.

12. A perfumed article in the form of a perfume, eau de toilette, after-shave lotion, fragrance, soap, bath or shower gel, deodorant or antiperspirant product, shampoo or any hair-care product, talc or powder of any nature, room spray, any cleaning product or detergent product, or fabric softener, characterized in that it comprises at least one methyl or ethyl ester of 2-hydroxy-3-methyl-3-butenoic acid.

13. Use of 2-hydroxy-3-butenoic acid esters as a perfuming ingredient.

14. Use according to claim 13, characterized in that the 2-hydroxy-3-butenoic acid ester has formula (I): 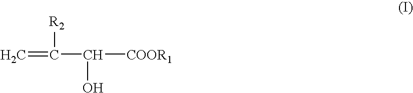
in which formula (I):
R₁ represents a hydrocarbon radical which may or may not be substituted, containing 1 to 40 carbon atoms, more particularly a linear or branched, saturated or unsaturated aliphatic acyclic radical; or a saturated or unsaturated, monocyclic or polycyclic carbocyclic or heterocyclic radical;
R₂ represents a saturated aliphatic radical with a low carbon condensation, preferably less than 4.

15. Use according to claim 13 or claim 14, characterized in that the 2-hydroxy-3-butenoic acid ester has general formula (Ia): 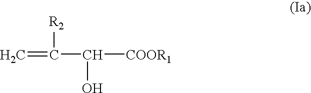
in which formula (Ia):
R₁ represents a linear or branched alkyl radical containing 1 to 12 carbon atoms, preferably 1 to 6; or a cycloalkyl radical preferably containing 6 carbon atoms, or an aralkyl radical containing 6 to 12 carbon atoms, preferably 7 or 8 carbon atoms;
R₂ represents a linear or branched alkyl radical containing 1 to 4 carbon atoms, preferably 1 or 2.

16. Use according to one of claims 13 to 15, characterized in that the 2-hydroxy-3-butenoic acid ester has general formula (I) in which R₁ represents an alkyl radical such as methyl, ethyl, n-propyl, isopropyl, butyl, isobutyl, sec-butyl, tert-butyl, amyl, isoamyl, n-hexyl; an allyl radical; a 2-hexenyl radical; a cyclohexyl radical; or a benzyl radical or a β-phenylethyl radical, and R₂ represents a methyl or ethyl radical.

17. Use according to one of claims 13 to 16, characterized in that the 2-hydroxy-3-butenoic acid ester is the methyl or ethyl ester of 2-hydroxy-3-methyl-3-butenoic acid.

18. Use of the methyl or ethyl ester of 2-hydroxy-3-methyl-3-butenoic acid as an autumnal note with a moist plum-tree evernia scent type associated with a nutty scent.
